(19) United States
(12) Patent Application Publication
(10) Pub. No.: US 2021/0373003 A1
(43) Pub. Date: Dec. 2, 2021

(54) IRREVERSIBLE AND COVALENT METHOD FOR IMMOBILIZING GLYCOPROTEIN

(71) Applicant: National Tsing Hua University, Hsinchu City (TW)

(72) Inventors: Chun-Cheng Lin, Hsinchu City (TW); Chen-Yu Fan, Hsinchu City (TW); Yu-Ju Chen, Hsinchu City (TW)

(73) Assignee: National Tsing Hua University, Hsinchu City (TW)

(21) Appl. No.: 16/921,958

(22) Filed: Jul. 7, 2020

(30) Foreign Application Priority Data
May 26, 2020 (TW) 109117564

(51) Int. Cl. G01N 33/543

(52) U.S. Cl. CPC G01N 33/54326; G01N 33/54346

(57) ABSTRACT

An irreversible and covalent method for immobilizing a glycoprotein includes the following steps. An organic boronic acid and a photoaffinity reagent are provided to contact a surface of a solid support, where the organic boronic acid is represented by R₁—ArB(OH)₂, —ArB(OH)₂ is a boronic acid group, and R₁ is a first cross-linking agent. The organic boronic acid is bound to the surface through the first cross-linking agent, and the photoaffinity reagent is bound to the surface through a second cross-linking agent R₂. Next, a glycoprotein is provided to contact the organic boronic acid, and the glycoprotein includes an Fc fragment. An alcohol group on a sugar chain of the Fc fragment and the boronic acid group of the organic boronic acid form an organic boronate ester to immobilize the glycoprotein. UV light irradiation is then performed, so that the photoaffinity reagent and the glycoprotein form a covalent cross-link.

### CROSS-REFERENCE TO RELATED APPLICATION

This application claims the priority benefit of Taiwan application serial no. 109117564, filed on May 26, 2020. The entirety of the above-mentioned patent application is hereby incorporated by reference herein and made a part of this specification.

### BACKGROUND

### Technical Field

The disclosure relates to an irreversible and covalent method for immobilizing a glycoprotein, and in particular, to an irreversible and covalent method for immobilizing a glycoprotein suitable for a complex analyte sample.

### Description of Related Art

An antibody may tightly and specifically bind to an epitope and therefore has been widely applied in biomedical technologies such as immunoaffinity separation, target treatment delivery, enzyme-linked immunosorbent assay (ELISA), and test arrays.

Nevertheless, antibody binding of an immobilized antibody is weak when the immobilized antibody is prepared through a conventional antibody cross-linking method, such as physical adsorption. Immobilization of antibody on the surface by random amide bond formation results in losing some of the antigen binding activity. Further, in a complex analyte sample (e.g., a blood sample), due to the presence of the alkaline substance, dissociation of antibody bound to the boronic acid may occur, and sensitivity of the subsequent antibody analysis is thus affected.

Based on the above, development of a method for immobilizing an antibody to resist dissociation, provide strong binding, and contribute to enhancement of detection sensitivity in a complex sample is an important issue.

### SUMMARY

The disclosure provides an irreversible and covalent method for immobilizing a glycoprotein through which an antibody may resist dissociation, provide strong binding, and contribute to enhancement of detection sensitivity in a complex sample.

An irreversible and covalent method for immobilizing a glycoprotein provided by the disclosure includes the following steps. An organic boronic acid and a photoaffinity reagent are provided to contact a surface of a solid support. The organic boronic acid is represented by R₁—ArB(OH)₂, —ArB(OH)₂ is a boronic acid group, and R₁ is a first cross-linking agent. The organic boronic acid is bound to the surface of the solid support through the first cross-linking agent R₁, and the photoaffinity reagent is bound to the surface of the solid support through a second cross-linking agent R₂. Next, a glycoprotein is provided to contact the organic boronic acid, and the glycoprotein includes an Fc fragment. An alcohol group on a sugar chain of the Fc fragment and the boronic acid group of the organic boronic acid form an organic boronate ester to immobilize the glycoprotein. UV light irradiation is then performed, so that the photoaffinity reagent and the glycoprotein form a covalent cross-link.

In an embodiment of the disclosure, the photoaffinity reagent is a diazirine compound.

In an embodiment of the disclosure, the solid support includes a nanoparticle.

In an embodiment of the disclosure, the first cross-linking agent R₁ is an organic linker containing an amine group at a terminal.

In an embodiment of the disclosure, the second cross-linking agent R₂ is an organic linker containing an amine group at a terminal.

In an embodiment of the disclosure, the glycoprotein includes an antibody.

In an embodiment of the disclosure, the glycoprotein includes an Fc-fusion glycoprotein including the Fc fragment.

In an embodiment of the disclosure, the irreversible and covalent method for immobilizing the glycoprotein is configured to detect an antigen in a blood sample.

To sum up, in the irreversible and covalent method for immobilizing the glycoprotein provided by the disclosure, the alcohol group on the sugar chain of the glycoprotein Fc fragment and the boronic acid group of the organic boronic acid form the organic boronate ester. UV light irradiation is further performed, so that the photoaffinity reagent and the glycoprotein form a covalent cross-link. In this way, the glycoprotein may exhibit dissociation resistance and strong binding and thereby contributes to enhancement of detection sensitivity and provides orientation in a complex sample (e.g., a blood sample).

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1A to FIG. 1D are schematic diagrams of an irreversible and covalent method for immobilizing a glycoprotein according to an embodiment of the disclosure.

FIG. 2 is a graph of fluorescence intensity measurement of stability of Example 1 and Comparative Example 1 in bovine blood with reaction time according to the disclosure.

FIG. 3A are analysis graphs of matrix-assisted laser desorption ionization-time of flight mass spectrometry (MALDI-TOF) for concentrated antigens in Example 1 and Comparative Example 2 according to the disclosure.

FIG. 3B is a graph of SAA/ISD (antigen concentration effect) ratio measurement of Example 1 and Comparative Example 2 according to the disclosure.

### DESCRIPTION OF THE EMBODIMENTS

The following describes the embodiments of the disclosure. Nevertheless, the embodiments are exemplary only, and the disclosure is not limited thereto.

In the specification, scopes represented by “a numerical value to another numerical value” are schematic representations in order to avoid listing all of the numerical values in the scopes in the specification. Therefore, the recitation of a specific numerical range covers any numerical value in the numerical range and a smaller numerical range defined by any numerical value in the numerical range, as is the case with any numerical value and a smaller numerical range thereof in the specification.

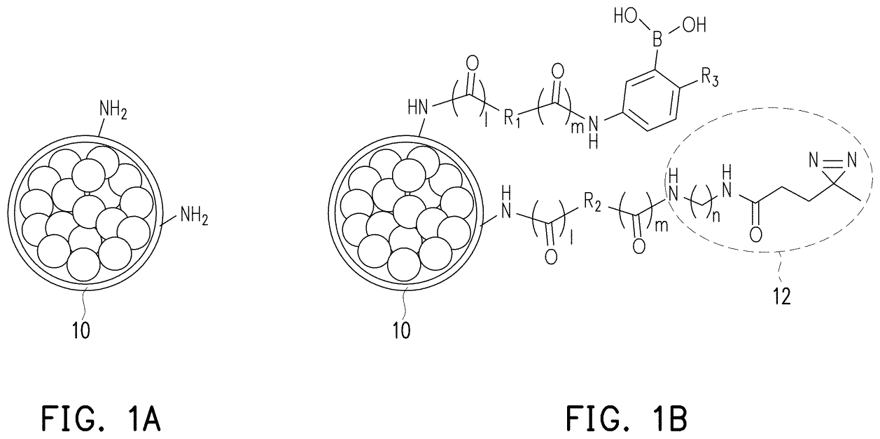

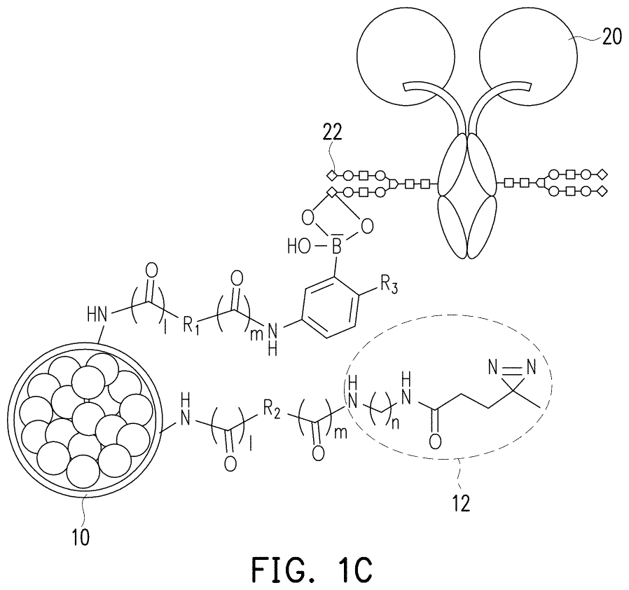

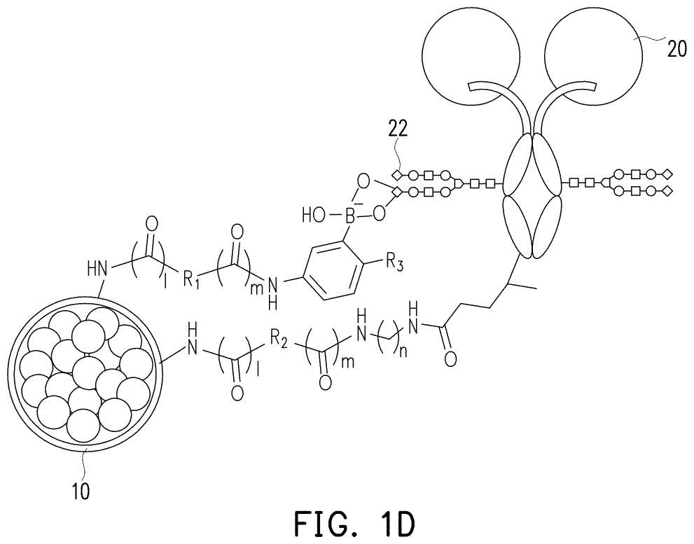

FIG. 1A to FIG. 1D are schematic diagrams of an irreversible and covalent method for immobilizing a glycoprotein according to an embodiment of the disclosure.

With reference to FIG. 1A, a solid support is provided, and the solid support may include a nanoparticle 10. The nanoparticle 10 is a magnetic nanoparticle having a size ranging from 2 nm to 800 nm. Next, with reference to FIG. 1B, an organic boronic acid and a photoaffinity reagent 12 are provided to contact a surface of the solid support (in this embodiment, the solid support is, for example, the nanoparticle 10). The organic boronic acid is represented by R₁—ArB(OH)₂, —ArB(OH)₂ is a boronic acid group, and R₁ is a first cross-linking agent. In this embodiment, the boronic acid may be represented by, for example, a chemical structure as follows, but the disclosure is not limited thereto:

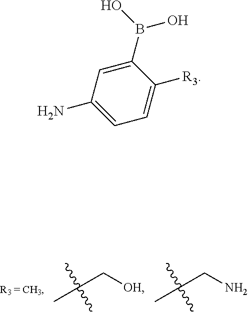

The organic boronic acid is bound to the surface of the solid support through the first cross-linking agent R₁ (in this embodiment, the solid support is, for example, the nanoparticle 10), and the first cross-linking agent R₁ may be an organic linker containing an amine group at a terminal.

With reference to FIG. 1B, the photoaffinity reagent 12 is, for example, a diazirine compound. In this embodiment, the photoaffinity reagent 12 may be represented by, for example, a chemical structure as follows:

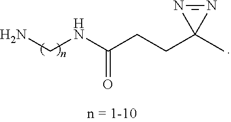

In addition, the photoaffinity reagent 12 may also be represented by, for example, a chemical structure as follows:

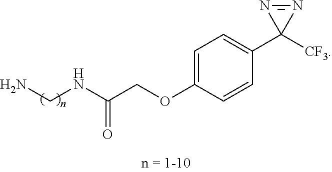

Note that the chemical structure and the number of n of the photoaffinity reagent 12 are merely exemplary for illustration, and the disclosure is not limited thereto. The photoaffinity reagent 12 is bound to the surface of the solid support (e.g., the nanoparticle 10 in this embodiment) through a second cross-linking agent R₂, and the second cross-linking agent R₂ may be an organic linker containing an amine group at a terminal.

With reference to FIG. 1B, the first cross-linking agent R₁ and the second cross-linking agent R₂ may be identical or may be different. The first cross-linking agent R₁ and the second cross-linking agent R₂ may include any carbon number and have diacid, diamine, or monoacid and monoamine structures at the ends. In this embodiment, the first cross-linking agent R₁ and the second cross-linking agent R₂ may be represented by a chemical structure of X—R—Y, for example, where R is an alkyl group, an alkenyl group, an alkynyl group, an aromatic group, or any combination of these four groups. Nevertheless, the carbon number provided herein is exemplary only, and the disclosure is not limited thereto. The X, Y, l, and m in the chemical structure of X—R—Y are defined as follows:

X═Y═NH₂, l=m=1;

X═Y═COOH, l=m=0;

X═NH₂, Y═COOH, l=0, m=1 or l=1, m=0; and

X═COOH, Y═NH₂, l=0, m=1 or l=1, m=0.

Note that the chemical structures and the definition of the parameters of the first cross-linking agent R₁ and the second cross-linking agent R₂ are exemplary only, and the disclosure is not limited thereto.

Next, with reference to FIG. 1C, a glycoprotein is provided to contact the organic boronic acid, and the glycoprotein includes an Fc fragment (crystallizable). In this embodiment, the glycoprotein is, for example, an antibody 20, but the disclosure is not limited thereto. An alcohol group on a sugar chain 22 of the Fc fragment of the antibody 20 and the boronic acid group of the organic boronic acid form an organic boronate ester to immobilize the glycoprotein (e.g., the antibody 20 in this embodiment) on the nanoparticle 10. To be more specific, the glycoprotein may be obtained by genetic engineering and glycoprotein engineering. Therefore, the glycoprotein may be an Fc-fusion glycoprotein and includes the Fc fragment having the sugar chain, so that the irreversible and covalent method for immobilizing the glycoprotein provided by the disclosure may be implemented.

Next, with reference to FIG. 1A, UV light irradiation is performed, so that the photoaffinity reagent and the glycoprotein (e.g., the antibody 20 in this embodiment) form a covalent cross-link. In this way, through the irreversible and covalent method for immobilizing the glycoprotein provided by the disclosure, when the glycoprotein is, for example, an antibody, binding of the antibody may be enhanced, so that an alkaline substance in a complex analyte sample is prevented from causing antibody dissociation. Therefore, the irreversible and covalent method for immobilizing the glycoprotein provided by the disclosure is suitable for further detecting an antigen in a blood sample, and sensitivity of a subsequent antibody analysis is thereby improved.

The irreversible and covalent method for immobilizing the glycoprotein provided by the foregoing embodiments are described in detail through experimental examples provided as follows. Nevertheless, the experimental examples below are not intended to limit the disclosure.

### Experimental Examples

The experimental example is provided as follow so as to prove that the irreversible and covalent method for immobilizing the glycoprotein provided by the disclosure may be used to enhance binding of an antibody, and that sensitivity of an antibody analysis may be effectively improved.

Note that since the irreversible and covalent method for immobilizing the glycoprotein is described in detail in the foregoing paragraphs, details of the irreversible and covalent method for immobilizing the glycoprotein provided below are omitted to simplify the description.

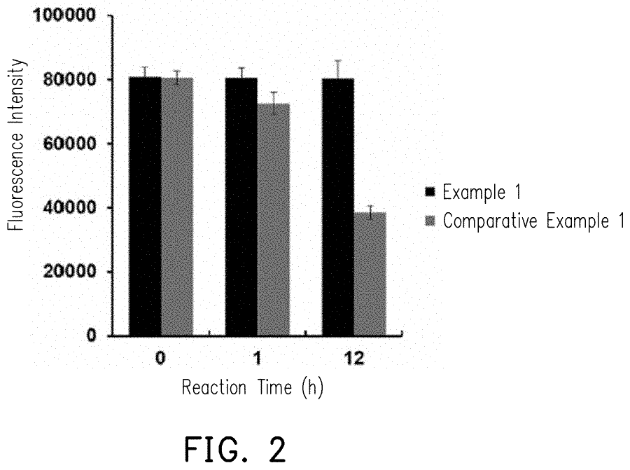

FIG. 2 is a graph of fluorescence intensity measurement of stability of Example 1 and Comparative Example 1 in bovine blood with reaction time according to the disclosure.

In FIG. 2, in Example 1, the magnetic nanoparticle to which the organic boronic acid and the photoaffinity reagent are bounded contacts an antibody mainly through the irreversible and covalent method for immobilizing the glycoprotein provided by this disclosure. The alcohol group on the sugar chain of the Fc fragment and the boronic acid group of the organic boronic acid form the organic boronate ester to immobilize the antibody. UV light irradiation is then performed, so that the photoaffinity reagent and the antibody form a covalent cross-link. In this way, the magnetic nanoparticle and the antibody of Example 1 may form oriented and irreversible binding. In Comparative Example, 1, the magnetic nanoparticle to which only the organic boronic acid is bound contacts the antibody. The alcohol group on the sugar chain of the Fc fragment and the boronic acid group of the organic boronic acid form the organic boronate ester to immobilize the antibody. In this way, only reversible binding of boronate ester is formed between the magnetic nanoparticle and the antibody of Comparative Example 1.

In a fluorescence measurement result of a binding analysis (binding assay) in FIG. 2, fluorescence intensity of Example 1 bounded to the antibody and fluorescence intensity of Comparative Example 1 bounded to the antibody are almost the same after Example 1 and Comparative Example 1 bound to the antibody are cultured in fetal bovine serum (FBS) for 1 hour. After being cultured in FBS for 12 hours, Example 1 bounded to the antibody does not show a significant change in fluorescence intensity, meaning that binding of Example 1 and the antibody is stabilized. In contrast, after being cultured in FBS for 12 hours, Comparative Example 1 bounded to the antibody show a significant reduction in fluorescence intensity by 50%, meaning that binding stability of Comparative Example 1 and the antibody decreases over time. According to the experimental result shown in FIG. 2, it can be seen that the irreversible and covalent method for immobilizing the glycoprotein provided by the disclosure may improve stability of binding of the nanoparticle and the antibody, and a certain degree of binding of the nanoparticle and the antibody may still be maintained even if time passes (after 12 hours). In particular, when the irreversible and covalent method for immobilizing the glycoprotein provided by the disclosure is applied to a complex analyte sample such as a blood sample, binding of the antibody may be stably and effectively maintained.

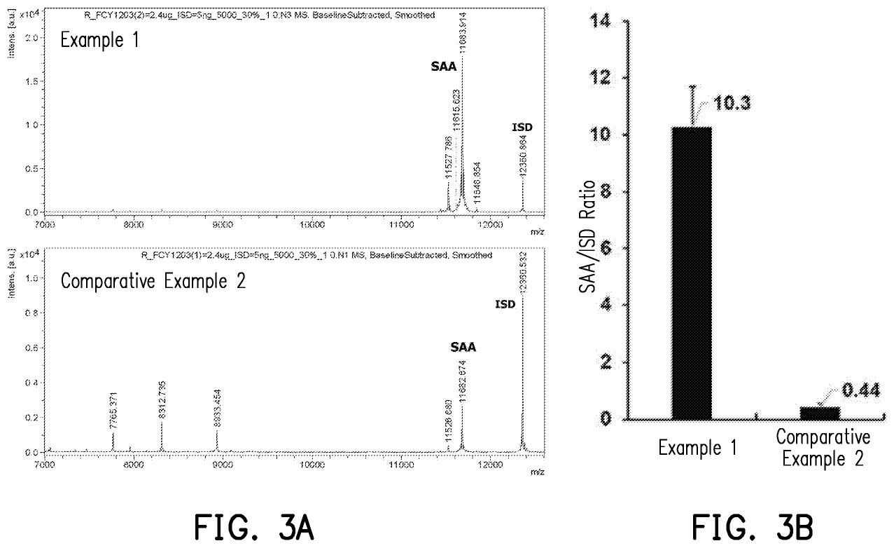

FIG. 3A are analysis graphs of matrix-assisted laser desorption ionixation-time of flight mass spectrometry (MALDI-TOF) for concentrated antigens in Example 1 and Comparative Example 2 according to the disclosure. FIG. 3B is a graph of SAA/ISD (antigen concentration effect) ratio measurement of Example 1 and Comparative Example 2 according to the disclosure.

In FIG. 3A and FIG. 3B, SAA is an acute phase plasma protein, and SAA detection in serum may provide a possible diagnosis of inflammation. ISD is an internal standard. In Example 1, the magnetic nanoparticle to which the organic boronic acid and the photoaffinity reagent are bounded contacts the antibody mainly through the irreversible and covalent method for immobilizing the glycoprotein provided by the disclosure. The alcohol group on the sugar chain of the Fc fragment and the boronic acid group of the organic boronic acid form the organic boronate ester to immobilize the antibody. UV light irradiation is then performed, so that the photoaffinity reagent and the antibody form a covalent cross-link. In this way, the magnetic nanoparticle and the antibody of Example 1 may form oriented and irreversible binding. In Comparative Example 2, the magnetic nanoparticle reacts with (3-aminopropyl)triethoxysilane (APTES) and a disuccinimidyl suberate (DSS) cross-linking agent. Therefore, the magnetic nanoparticle and the antibody of Comparative Example 2 may not form oriented and irreversible binding. As shown in FIG. 3A, high specificity for SAA binding is presented in Example 1. As shown in FIG. 3B, the SAA/ISD ratio of Example 1 is approximately 20 times the SAA/ISD ratio of Comparative Example 2. Therefore, it can be seen that the specificity of antibody binding may be maintained through the irreversible and covalent method for immobilizing the glycoprotein provided by the disclosure, and further, sensitivity of the subsequent antigen detection may be effectively improved.

In view of the foregoing, in the irreversible and covalent method for immobilizing the glycoprotein provided by the disclosure, the alcohol group on the sugar chain of the glycoprotein Fc fragment and the boronic acid group of the organic boronic acid form the organic boronate ester. UV light irradiation is further performed, so that the photoaffinity reagent and the glycoprotein form a covalent cross-link. In this way, oriented and irreversible binding may be formed between the nanoparticle and the glycoprotein, and high binding specificity is also provided. Therefore, in a complex sample (e.g., a blood sample), the glycoprotein may exhibit dissociation resistance and strong binding and thereby contributes to enhancement of detection sensitivity and provides orientation.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. An irreversible and covalent method for immobilizing a glycoprotein, comprising:
providing a solid support;
providing an organic boronic acid and a photoaffinity reagent to contact a surface of the solid support, the organic boronic acid represented by R₁—ArB(OH)₂, wherein —ArB(OH)₂ is a boronic acid group, R₁ is a first cross-linking agent, the organic boronic acid is bound to the surface of the solid support through the first cross-linking agent, and the photoaffinity reagent is bound to the surface of the solid support through a second cross-linking agent R₂;
providing a glycoprotein to contact the organic boronic acid, the glycoprotein comprising an Fc fragment (crystallizable), wherein an alcohol group on a sugar chain of the Fc fragment and the boronic acid group of the organic boronic acid form an organic boronate ester to immobilize the glycoprotein; and
performing UV light irradiation is performed, so that the photoaffinity reagent and the glycoprotein form a covalent cross-link.

2. The irreversible and covalent method for immobilizing the glycoprotein according to claim 1, wherein the photoaffinity reagent is a diazirine compound.

3. The irreversible and covalent method for immobilizing the glycoprotein according to claim 1, wherein the solid support comprises a nanoparticle.

4. The irreversible and covalent method for immobilizing the glycoprotein according to claim 1, wherein the first cross-linking agent R₁ is an organic linker containing an amine group at a terminal.

5. The irreversible and covalent method for immobilizing the glycoprotein according to claim 1, wherein the second cross-linking agent R₂ is an organic linker containing an amine group at a terminal.

6. The irreversible and covalent method for immobilizing the glycoprotein according to claim 1, wherein the glycoprotein comprises an antibody.

7. The irreversible and covalent method for immobilizing the glycoprotein according to claim 1, wherein the glycoprotein comprises an Fc-fusion glycoprotein comprising the Fc fragment.

8. The irreversible and covalent method for immobilizing the glycoprotein according to claim 1, wherein the irreversible and covalent method for immobilizing the glycoprotein is configured to detect an antigen in a blood sample.
